(19) United States
(12) Patent Application Publication
(10) Pub. No.: US 2015/0297609 A1
(43) Pub. Date: Oct. 22, 2015

(54) STERILE OPHTHALMIC PHARMACEUTICAL SUSPENSIONS

(71) Applicants: Mandar V. SHAH, Haryana (IN); SENTISS PHARMA PVT. LTD., Haryana (IN)

(72) Inventors: Mandar V. Shah, Haryana (IN); Deepak Bahri, Haryana (IN)

(73) Assignee: SENTISS PHARMA PVT. LTD., Haryana (IN)

(21) Appl. No.: 14/646,214

(22) PCT Filed: Dec. 19, 2013
(86) PCT No.: PCT/US13/76556

§ 371 Date: May 20, 2015

Related U.S. Application Data
(60) Provisional application No. 61/746,357, filed on Dec. 27, 2012.

(51) Int. Cl. A61K 31/542; A61K 9/00

(52) U.S. Cl. CPC A61K 31/542; A61K 9/0048

(57) ABSTRACT

An aqueous suspension of a carbonic anhydrase inhibitor such as brinzolamide as well as methods of preparation thereof, are disclosed. The suspension comprises an organic polar solvent such as dimethyl sulfoxide and/or a non-aromatic non-ionic polyethoxylated surfactant such as polyethoxylated castor oil and/or or polysorbate 80. Methods include precipitating brinzolamide from the heated composition. Methods also include sonication of the suspension to reduce brinzolamide particle size.

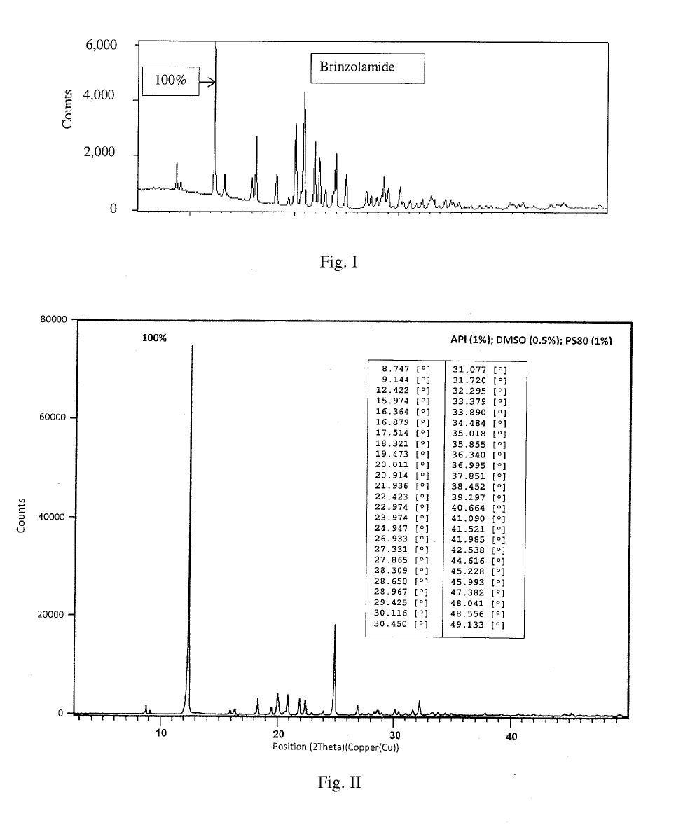

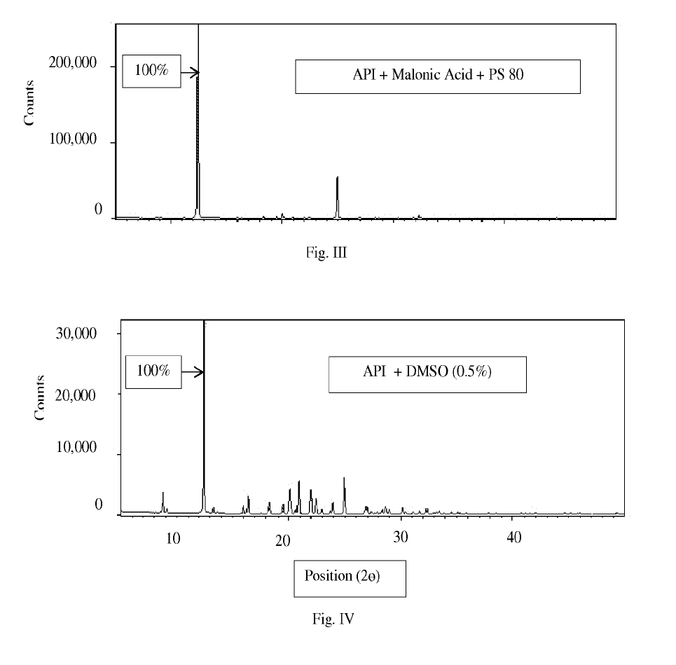

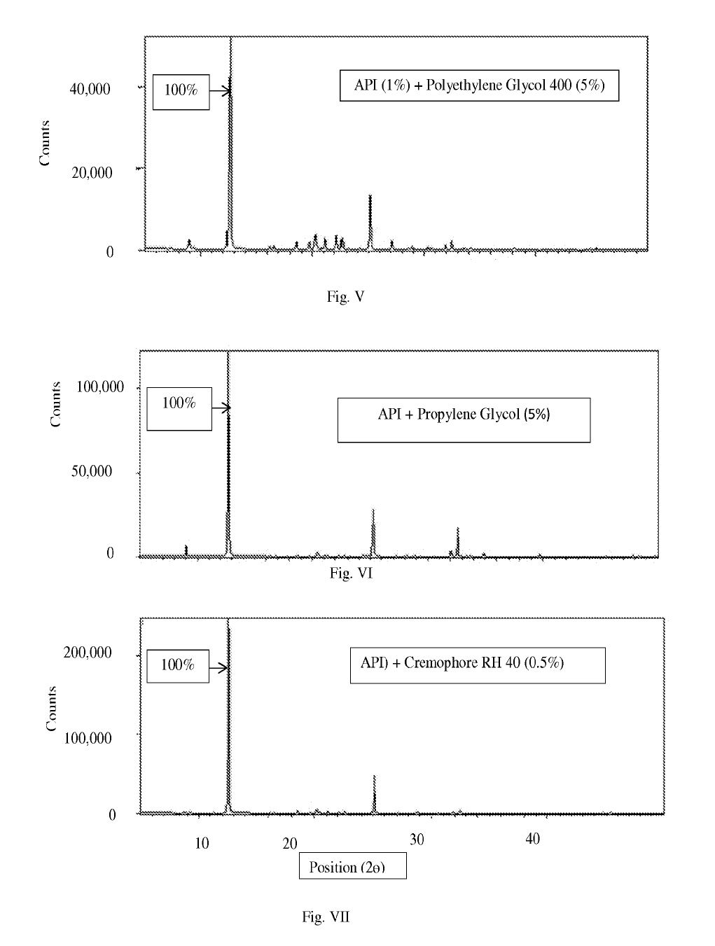

### CROSS-REFERENCE TO RELATED APPLICATION

This application claims priority to U.S. provisional application 61/746,357, filed Dec. 27, 2012, the contents of which are incorporated herein by reference in their entirety.

### FIELD OF THE INVENTION

The invention relates to sterile, ophthalmic pharmaceutical suspensions comprising active ingredient(s) such as carbonic anhydrase inhibitors (CAIs) like brinzolamide and/or other pharmaceutically acceptable excipients.

### BACKGROUND

Brinzolamide is a carbonic anhydrase inhibitor used to lower intraocular pressure in patients with ocular hypertension or open-angle glaucoma. Brinzolamide is chemically (R)-(+)-4-ethylamino-2-(3-methoxypropyl)-3,4-dihydro-2H-thieno[3,2-e]-1,2-thiazine-6-sulfonamide-1,1-dioxide and has the empirical formula C₁₂H₂₁N₃O₅S₃. Brinzolamide has a molecular weight of 383.5. A commonly used crystal form of the drug has a melting point of about 131° C.

This compound is disclosed in U.S. Pat. No. 5,378,703 (Dean, et al.). The compound is also disclosed in European patent EP 527801. U.S. Pat. No. 6,071,904 discloses processes for preparation of brinzolamide ophthalmic composition.

Brinzolamide ophthalmic suspension is developed and marketed by Alcon Laboratories Inc. in United States under the brand name Azopt® (brinzolamide ophthalmic suspension 1%). Brinzolamide is indicated for lowering elevated intra-ocular pressure (IOP) in patients with open-angle glaucoma or ocular hypertension (OHT).

Various methods have been disclosed in the prior for the preparation of brinzolamide ophthalmic suspension. International patent application WO 98/25620 teaches that conventional sterilization methods cannot be employed in the manufacture of suspensions comprising brinzolamide since the compound recrystallizes as large needle-shaped crystals, upon cooling, after autoclaving.

According to WO 98/25620, dry heat sterilization is also not suitable, since it causes melting of the material, whereas sterilization by ethylene oxide and gamma irradiation introduces unacceptable degradation products.

EP0941094 discloses a process for making brinzolamide suspension by autoclaving of concentrated slurry of brinzolamide and tyloxapol; or brinzolamide and Triton X in milling bottle, and ball milling of the hot slurry after autoclaving, and then adding the slurry to the rest of the ingredients. It should be noted here that high temperatures and pressures of autoclave will dissolve brinzolamide. Later, when autoclaving is complete, upon cooling brinzolamide precipitates as large shaped crystals, having particle size of 1000 to 5000 μm. However, inclusion of tyloxapol and/or Triton X in the slurry allows the crystals to break up easily by ball milling. Brinzolamide cannot be administered as these large needle shaped crystals, as they will damage the eyes. Fine particles (small enough to reduce or preferably prevent eye irritation) are preferred. Hence, precipitated brinzolamide crystals need to be milled to reduce their particle size.

Thus, the reference discloses autoclaving of the slurry of brinzolamide and surfactant and further ball milling the slurry. However, the drawback associated with this method is that it requires a milling bottle in which the slurry of brinzolamide could initially be autoclaved and then ball milled for further size reduction of needle shaped crystals of brinzolamide that are formed during autoclaving.

Dry heat sterilization causes melting of the material. Sterilization by ethylene oxide introduces unacceptable degradation products and residues, and sterilization by gamma irradiation of micronized material produces degradation products unacceptable for regulatory filing.

The majority of the suspensions disclosed in the references faced the problem of crystallization and agglomeration of active ingredients during preparation as well as during storage. Crystallization or agglomeration of active leads to non-uniformity of dose, difficulty of administration, irritation to eye due to large drug particles and/or any ocular adverse effect due to high drug concentration.

In most cases crystallization of active ingredients useful for ophthalmic use like carbonic anhydrase inhibitor, or others actives, occurs during preparation. Sterilization by autoclaving at temperature of 121° C. and 115 lbs of pressure leads to increase in solubility of the actives in the preparation and at that temperature brinzolamide goes into solution. However, upon cooling, brinzolamide precipitates as needle shaped crystals. These needle-shaped crystals are difficult to break and suspend. In different references either tyloxapol is used in solution so that the crystals are easier to break or special equipment such as ball mill and/or jet mill is used to break the large needle-shaped crystals.

There remains a need for a method to prepare a sterile, ophthalmic pharmaceutical aqueous suspension, of an active ingredient with low aqueous solubility (such as brinzolamide) wherein, after autoclaving, the active ingredient precipitates with a particle size less than about 50 microns; preferably less than about 30 microns, and/or precipitates in a form other than needle-shaped crystals, e.g., in a different crystal habit or in a fine crystalline form, or partially amorphous (or equivalently, partially crystalline) form.

There remains a need for a method to reduce particle size of an aqueous suspension of an active ingredient with low aqueous solubility (such as brinzolamide) using more economical method other than ball or jet milling, e.g., using sonication and/or simple stirring or light homogenization.

So, the inventors of present inventors had done various experiments by adding different innocuous ingredients such as but not limited to, dimethyl sulfoxide (DMSO) or Cremophor® RH 40 or polysorbate 80 or polyethylene glycol 400, propylene glycol, several acids such as ascorbic acid, malonic acid or combination of these ingredient(s). It was surprisingly found by the inventors of the present invention that by the addition of certain ingredients such as dimethyl sulfoxide (DMSO) and polysorbate 80 leads to increased crystallinity of the brinzolamide. Furthermore, out of the experiments as carried by the inventors of the present invention, they surprisingly found that a formulation that comprises dimethyl sulfoxide (DMSO) and polysorbate 80 along with brinzolamide had a marked increase in the crystallinity by about 25 times to about 30 times. This is demonstrated by X-ray diffractogram shown in FIG. II.

Thus, without being bound by theory, it is believed that the present invention surprisingly stabilizes the brinzolamide crystal structure by further enhancing its crystallinity, thereby reducing the free energy.

Furthermore, the present invention also paves way to stable formulations comprising brinzolamide which may lead to an enhanced shelf life with reduced, little or no problems of resuspendability as associated with the suspension formulations due to lower free energy in the system.

The inventors of present invention had furthermore found that fine crystals of brinzolamide were formed by using more economical methods such as by sonication technique and/or simple stirring or light homogenization, rather than ball or jet milling, wherein thus the particle size obtained in the present invention was below than about 20 microns as shown in Table 3.

### SUMMARY OF THE INVENTION

The present invention provides a process of preparing a sterile, ophthalmic pharmaceutical suspension, wherein the active ingredient brinzolamide, precipitates during cooling after autoclaving in small particles, preferably not comprising needle-like crystals, preferably in a partially amorphous or highly crystalline form. The brinzolamide precipitate preferably has an average particle size less than about 150 microns, which can be further reduced in size easily, e.g., by sonication. The final particle size is preferably less than 30 microns. After precipitation, the suspension may be made by simple mixing or light homogenization and further reduced in size by, e.g., sonication.

The present invention further provides a process, wherein the precipitated brinzolamide (preferably crystalline) as a part, is formed by the use of ophthalmically acceptable ingredients such as dimethyl sulfoxide (DMSO), polyethoxylated hydrogenated or non-hydrogenated castor oil (e.g., Cremophor), polysorbate 80, or a combination of these ingredient(s).

This present invention additionally provides a process for preparation of a suspension, the process being efficient, economic, and feasible for commercial scale preparation and in which the suspension does not comprise an alkyl aryl polyether alcohol, and preferably does not comprise tyloxapol or Triton X. Tyloxapol and Triton X are both alkyl aryl polyether alcohols; compositions and methods of the present invention preferably do not comprise such surfactants. Preferably, the process of the present invention does not require the use of specific equipment such as ball mill and/or jet mill, though such equipment and methods may be used if desired.

An objective of the present invention is to provide a process of preparing a sterile, ophthalmic pharmaceutical suspension, wherein the precipitates of brinzolamide formed during cooling after autoclaving is further reduced in a partially amorphous, crystalline, or highly crystalline form, preferably by sonication technique and/or simple stirring or light homogenization, preferably reducing the particle size to less than about 50 microns; preferably less than about 30 microns or more preferably below 10 microns.

Further sonication is a simple, cost effective and easily reproducible method used for particle size reduction.

Further the invention provides a process which ameliorates one or more drawbacks of the reference cited processes.

The sterile, ophthalmic pharmaceutical brinzolamide suspensions as prepared in the present invention are useful in the treatment of conditions treatable with brinzolamide, e.g., elevated intraocular pressure in persons suffering from ocular hypertension or primary open angle glaucoma.

The inventors of the present invention have developed a process for preparation of sterile, ophthalmic pharmaceutical suspension wherein the process does not contain tyloxapol, or Triton X or the process does not require the use of specific equipment's such as ball mill and/or jet mill.

An objective of the present invention is to provide a sterile, ophthalmic pharmaceutical suspension and process of making such suspension comprising active ingredient(s) such as carbonic anhydrase inhibitors (CAIs) like brinzolamide, wherein the suspension does not contain tyloxapol, or Triton X or the process does not require the use of specific equipment's such as ball mill and/or jet mill.

Another objective of the present invention is to provide a process of preparing a sterile, ophthalmic pharmaceutical suspension, wherein the active ingredient brinzolamide, precipitates during cooling after autoclaving in a fine particulate form, preferably crystalline or partially amorphous form, such that the reduced average particle size of brinzolamide is less than about 100 microns, which can be further reduced in size below 50 microns; preferably less than about 30 microns by simple sonication and/or mixing or light homogenization.

Another objective of the present invention is to provide a process, wherein the precipitated (preferably crystalline) brinzolamide as a part, is formed by the use of pharmaceutically acceptable (innocuous) ingredients such as dimethyl sulfoxide (DMSO) or Cremophor® RH 40 or polysorbate 80 or polyethylene glycol 400, propylene glycol, several acids such as ascorbic acid, malonic acid, nicotinic acid or combination of these ingredient(s).

Another objective of the invention is to provide a process for preparation of sterile, crystalline brinzolamide ophthalmic suspension, the process being efficient, economic, and feasible for commercial scale preparation and which does not involve tyloxapol, or Triton X or the use of any special equipment's such as ball mill and/or jet mill.

Another object of the invention is to provide a process which ameliorates one or more drawbacks of the reference cited processes.

The present invention provides an aqueous composition comprising water, particles of brinzolamide, a polar organic solvent, and a non-ionized non-aromatic ethoxylated surfactant. The present invention also provides a sterile, ophthalmic pharmaceutical suspension and process of making such suspension comprising active ingredient(s) such as carbonic anhydrase inhibitors (CAIs) like brinzolamide, wherein the suspension does not contain tyloxapol, or Triton X or the process does not require the use of special equipment's such as ball mill and/or jet mill.

The present invention also provides a process for preparing a sterile, ophthalmic pharmaceutical suspension, the process comprising a) preparing a slurry comprising brinzolamide and a surfactant; b) adding to the brinzolamide slurry a solvent(s); c) precipitating a portion of brinzolamide slurry as a fine particulate (preferably partially amorphous or crystalline form) during cooling after autoclaving; d) preparing sterile polymer slurry comprising polymer, tonicity agent and preservative; e) adding said suspension vehicle of step (c) to said slurry of step (b) to obtain a suspension and making up the volume with water; and e) stirring the said suspension of step (d) while cooling. Also included are compositions made by the process.

The present invention also provides a method of making an aqueous brinzolamide suspension comprising combining water, brinzolamide, a polar organic solvent, and a non-ionized non-aromatic ethoxylated surfactant to form a first composition, heating the first composition to sterilize or dissolve the brinzolamide to form a second composition, and precipitating brinzolamide from the second composition to form a third composition, wherein the precipitated brinzolamide in the third composition has an average particle size less than 100 pm. Also included is a method which further comprises sonicating the third composition to form a fourth composition, wherein the brinzolamide in the fourth composition has an average particle size less than 50 pm. Also included is a method that further comprises combining the third or fourth composition with one or more of a polymer, an isotonicity agent, a viscosity enhancing agent, a buffer, a pH adjusting agent, an antioxidant, a chelating agent, and a preservative. Also included are compositions made by the inventive methods.

The present invention also provides a method of treating elevated intraocular pressure in patients with ocular hypertension or open-angle glaucoma in a subject in need of such treatment, the method comprising administering to a subject in need thereof a suspension wherein the suspension does not contain tyloxapol, or Triton X or the process does not require the use of special equipment's such as ball mill and/or jet mill.

The present invention also provides a method of treating an ocular condition treatable with brinzolamide, in a person in need thereof, comprising administering to the person an ophthalmic composition comprising water, an effective amount of particles of brinzolamide, a polar organic solvent, and a non-ionized non-aromatic ethoxylated surfactant.

Inventive processes and methods preferably include those wherein the active ingredient brinzolamide, precipitates during cooling after autoclaving in a fine crystalline form, such that the reduced average particle size of brinzolamide is preferably less than about 100 microns, which can be preferably further reduced in size below 50 microns; preferably less than about 30 microns by sonication and/or simple mixing or light homogenization. Preferably, during cooling at least 10% of the brinzolamide precipitates in a fine crystalline form, so that there is preferably a reduction in therapeutically effective dose of brinzolamide. Preferably, the process does not involve the use of special equipment such as ball mill and/or jet mill. Preferably, the surfactant comprises at least one of polysorbate 80 and Cremophor® RH 40. Preferably, the solvent comprises dimethyl sulfoxide (DMSO).

Preferably, the inventive compositions include sterile, ophthalmic pharmaceutical suspensions prepared by the inventive process or method, and comprise brinzolamide in an amount of from about 0.01% to 5.0% by weight.

The inventive processes and methods preferably include preparing a sterile, ophthalmic pharmaceutical suspension, wherein, preferably at least a portion of the active ingredient brinzolamide precipitates as fine crystalline form, and/or wherein preferably the precipitated crystalline brinzolamide as a part, is formed by the use of one or more of dimethyl sulfoxide (DMSO), Cremophor® RH 40, and polysorbate 80, or a combination of one or more of these ingredient(s). Preferably, in the inventive methods and processes, the suspension is free of tyloxapol and/or Triton X-100.

Preferably, the particles of brinzolamide have an average size less than 100, more preferably less than 50 μm. Preferably, the polar organic solvent comprises DMSO. Preferably, the surfactant comprises at least one of polysorbate 80 or polyethoxylated castor oil. Preferably, the composition is a sterile suspension suitable for ophthalmic use. Preferably, the composition comprises one or more of a polymer, an isotonicity agent, a viscosity enhancing agent, a buffer, a pH adjusting agent, an antioxidant, a chelating agent, and a preservative. Preferably, the composition does not comprise tyloxapol or Triton X.

Preferably, the heating comprises autoclaving. Preferably, neither tyloxapol nor Triton X are added prior to precipitation.

Preferably, the ophthalmic composition is a sterile suspension suitable for ophthalmic use. Preferably, the condition treatable with brinzolamide includes elevated intraocular pressure. Preferably, the brinzolamide is administered one drop per eye, 1-3 times per day, at a concentration of 1-15 mg brinzolamide per ml of the ophthalmic composition. Preferably, the particles of brinzolamide have an average size less than 20 μm. Preferably, the ophthalmic composition further comprises one or more of a polymer, an isotonicity agent, a viscosity enhancing agent, a buffer, a pH adjusting agent, an antioxidant, a chelating agent, and a preservative. Preferably, the ophthalmic composition does not comprise tyloxapol or Triton X.

### BRIEF DESCRIPTION OF THE DRAWINGS

FIG. I is a diffractrogram of brinzolamide formulation comprising pure API.

FIG. II is a diffractrogram of brinzolamide formulation comprising API (1%): DMSO (0.5%): PS80 (1%).

FIG. III is a diffractrogram of brinzolamide formulation comprising API: Malonic acid: PS 80.

FIG. IV is a diffractrogram of brinzolamide formulation comprising API: DMSO.

FIG. V is a diffractrogram of brinzolamide formulation comprising API: PEG 400.

FIG. VI is a diffractrogram of brinzolamide formulation comprising API: propylene glycol.

FIG. VII is a diffractrogram of brinzolamide formulation comprising API: Cremophore RH 40.

### DETAILED DESCRIPTION OF THE INVENTION

The present invention provides a sterile, ophthalmic pharmaceutical suspension and process of making such suspension comprising active ingredient(s) such as carbonic anhydrase inhibitors (CAIs) like brinzolamide, wherein the suspension does not contain tyloxapol, or Triton X or the process does not require the use of specific equipment's such as ball mill and/or jet mill.

The present invention provides a process of preparing a sterile, ophthalmic pharmaceutical suspension, wherein the active ingredient brinzolamide, precipitates during cooling after autoclaving in a fine particulate form, e.g., in a partially amorphous or crystalline form, such that the reduced average particle size of brinzolamide is less than about 100 microns, which can be preferably further reduced in size below 50 microns; preferably less than about 30 microns by simple sonication and/or mixing or light homogenization.

According to one aspect of the present invention there is provided a process for preparing a sterile, ophthalmic pharmaceutical suspension, the process comprising:
a) preparing a slurry comprising brinzolamide and a surfactant;
b) adding to the brinzolamide slurry a solvent(s);
c) precipitating a portion of brinzolamide slurry as a fine particulate (e.g., crystalline) form during cooling after autoclaving;
d) optionally preparing sterile polymer slurry comprising polymer, tonicity agent and preservative;
e) optionally combining said suspension vehicle of (c) with said slurry of (b) to obtain a suspension; and making up the volume with water;
f) optionally stirring the said suspension of step (d) while cooling.

According to one aspect of the present invention, there is provided a process for preparing a sterile, ophthalmic pharmaceutical suspension, the process comprising:
a) obtaining a composition comprising brinzolamide, water, a non-ionic non-aromatic surfactant, and a polar organic solvent;
b) heating the composition to obtain a sterilized composition with the brinzolamide dissolved therein;
c) precipitating brinzolamide from the sterilized composition; and
d) optionally reducing the brinzolamide particle size, preferably by sonication.

Further in one aspect of the present invention, the inventors of the present invention have conducted extensive studies and add ophthalmically acceptable ingredients such as dimethyl sulfoxide (DMSO) or Cremophor® RH 40 or polysorbate 80 or combination of these ingredients that will alter the crystal formation of brinzolamide, when it precipitates from the solution on cooling.

Furthermore the present invention provides a process wherein, at least a portion of the active ingredient brinzolamide precipitates in a fine crystalline form, wherein the precipitated crystalline brinzolamide as a part, is formed by the use of innocuous ingredients such as dimethyl sulfoxide (DMSO) or Cremophor® RH 40 or polysorbate 80 or combination of these ingredient(s).

The precipitated brinzolamide is preferably in partially amorphous or crystalline form, preferably in crystalline form, more preferably in highly crystalline form. The particles are preferably in the form of fine particles. More preferably the fine particulate has an average particle size of less than 100 μm, more preferably less than 50 μm. The particle size can be reduced further by sonication to an average particle size less than 50 am, more preferably less than 30 pm or 20 pm. Any method can be used to determine particle size distribution. One preferred method is to use a light scattering or Coulter counter method. It is preferred that 90% of the particles (D(0.9)) be less than 15 pm, more preferably, D(0.9) is in the range of 10-15 pm. It is preferred that 100% of the particles (D(1)) be less than 30 pm, more preferably less than about 20 pm.

In a preferred embodiment of the present invention, there is provided a sterile, ophthalmic pharmaceutical suspension prepared by the process as described herein comprising brinzolamide in an amount from about 0.01% to 5.0% by weight and/or pharmaceutically acceptable excipients.

In a more preferred embodiment of the present invention, the pharmaceutically acceptable excipients are selected from but not limited to at least one surfactant, at least one isotonicity agent, at least one viscosity enhancing agent and at least one preservative are added in the form of at least one sterile solution to the non-sterile brinzolamide.

According to one embodiment, the present invention relates to provide a sterile, ophthalmic pharmaceutical suspension and process of making such suspension comprising active ingredient(s) such as carbonic anhydrase inhibitors (CAIs) like brinzolamide, wherein the suspension does not contain tyloxapol, or Triton X or the process does not require the use of specific equipment's such as ball mill and/or jet mill.

One of the embodiments of the present invention is to provide a process of preparing a sterile, ophthalmic pharmaceutical suspension, wherein the active ingredient brinzolamide, precipitates during cooling after autoclaving in a particulate form, preferably fine particulate form, which can be partially amorphous or crystalline. The reduced average particle size of brinzolamide is preferably less than about 100 microns and can be preferably further reduced in size below 50 microns; preferably less than about 30 microns by simple sonication and/or mixing or light homogenization.

In one of the aspect of the present invention, the inventors of the present invention have conducted extensive studies and add innocuous ingredients such as dimethyl sulfoxide (DMSO) or Cremophor® RH 40 or polysorbate 80 or combination of these ingredients that will alter the crystal formation of brinzolamide, when it precipitates from the solution on cooling. Further, in the present invention at least a portion of the active ingredient brinzolamide precipitates as a fine particulate form. This appears to be unlike when tyloxapol is present as in prior inventions, wherein brinzolamide precipitates as crystalline material with large particle size.

Another embodiment of the present invention is to provide a process of preparing a sterile, ophthalmic pharmaceutical suspension, wherein during cooling at least 10% of the brinzolamide precipitates in a fine particulate (preferably crystalline) form, which may permit a reduction in therapeutically effective dose of brinzolamide.

Another embodiment of the present invention is to provide a process, wherein the precipitated particulate (preferably crystalline) brinzolamide as a part, is formed by the use of pharmaceutically acceptable ingredients such as dimethyl sulfoxide (DMSO) or Cremophor® RH 40 or polysorbate 80 or combination of these ingredient(s).

In one of the feature, the present invention provides a process for preparation of sterile, crystalline brinzolamide ophthalmic suspension, the process being efficient, economic, and feasible for commercial scale preparation and which does not involve tyloxapol, or Triton X or the use of any special equipment's such as ball mill and/or jet mill.

Another embodiment of the present invention is to provide a process which ameliorates one or more drawbacks of the reference cited processes.

The sterile, ophthalmic pharmaceutical suspensions as prepared in the present invention are useful in the treatment of elevated intraocular pressure in persons suffering from ocular hypertension or primary open angle glaucoma.

According to one embodiment of the present invention, there is provided a sterile ophthalmic suspension prepared by the process as described herein comprising brinzolamide in an amount from 0.01% to 5.0% by weight.

According to one of the embodiment, the present invention provides brinzolamide ophthalmic suspensions comprising: the active ingredient brinzolamide, polyoxyl 40 hydrogenated castor oil (e.g., Cremophor® RH 40); Carbomer® 974 P; mannitol, sodium chloride, edetate disodium, benzalkonium chloride; sodium hydroxide and/or hydrochloric acid (to adjust the pH) wherein the said ophthalmic suspension is prepared by the process as described herein.

According to a preferred embodiment, the present invention provides brinzolamide ophthalmic suspensions comprising: the active ingredient brinzolamide, polysorbate 80; dimethyl sulfoxide (DMSO); Carbomer® 974 P; mannitol, sodium chloride, edetate disodium, benzalkonium chloride; sodium hydroxide and/or hydrochloric acid (to adjust the pH) wherein the ophthalmic suspension is prepared by the process as described herein.

In a more preferred embodiment of the present invention, the pharmaceutically acceptable excipients are selected from but not limited to at least one polymer, at least one surfactant, at least one isotonicity agent, at least one viscosity enhancing agent, at least one solvent, at least one buffer, at least one pH adjusting agents, at least one antioxidants, at least one chelating agents and at least one preservative.

An alkyl aryl polyether alcohol surfactant is preferably not included during precipitation of brinzolamide. It will be appreciated, however, that such surfactants, including tyloxapol and/or Triton X, can be added to the inventive compositions after brinzolamide precipitation without departing from the scope of the invention.

In one of the embodiments, the polymers that may be used is selected from the group consisted of, but are not limited to Carbomer® such as Carbomer 974 P, povidone, hydroxypropylmethylcellulose, hydroxypropylcellulose, hydroxyethylcellulose and mixtures thereof, wherein the said polymers may be used in amount from 0.1% to 5.0%.

In one of the embodiments, the preservatives that may be used is selected from the group consisted of, but are not limited to benzethonium chloride, phenyl ethanol, phenyl propanol, phenyl mercuric acetate, phenyl mercuric nitrate, phenyl mercuric borate, chlorhexidine acetate or gluconate, cetrimide, chlorocresol, benzoic acid, benzyl alcohol, butylparaben, propylparaben, methylparaben, chlorobutanol, phenoxyethanol, sodium methyl paraben, sodiumpropyl paraben, thimerosal, benzalkonium chloride and mixtures thereof, wherein the said preservatives may be used in an amount from 0.005% to 0.5%.

In another embodiment, the surfactants that may be used is selected from the group consisted of, but are not limited to sodium lauryl sulfate, docusate sodium, polyoxyalkyl ethers, polyoxylalkyl phenyl ethers, polyoxyl 40 hydrogenated castor oil (Cremophor® RH 40), polyoxyl 40 stearates, polyoxy hydrogenated castor oil, polyoxy sorbitan esters, sorbitan esters, polysorbates, polyoxyl 35 castor oil, sorbitan monolaureates, poloxamer and mixtures thereof, wherein the said surfactants may be used in amount from 0.001% to 15%, preferably 0.01% to 0.5%.

In one of the embodiments, the tonicity agents that may be used is selected from the group consisted of, but are not limited to mannitol, dextrose, glycerin, potassium chloride, sodium chloride and mixtures thereof, wherein the tonicity agents may be used in amount from about 1.0% to 5.0% or is added in such an amount that makes the osmotic pressure of the composition identical to that of tears.

Any effective agent for reducing particle size may be used. Preferred agents for reducing brinzolamide particle size include polar organic solvents and/or organic acids. In a preferred embodiment, the solvent, preferably a polar organic solvent, is selected from the group consisting of, but not limited to, aliphatic and aromatic alcohols, ethanol, dimethyl sulfoxide (DMSO), dimethyl acetamide, ethoxydiglycol, isopropyl myristate, triacetin, polyethylene glycols, propylene glycol, and combinations thereof. In another preferred embodiment, the organic acid may be selected from the group consisting of, but not limited to, several acids such as ascorbic acid, malonic acid, nicotinic acid or a combination of these ingredients. Combinations of particle size reducing agents (e.g., polar organic solvent and acid) are also included.

In another embodiment, the viscosities enhancing agents that may be used is selected from the group comprising of, but are not limited to, carboxymethylcellulose sodium, hydroxyethyl cellulose, hydroxypropyl cellulose, hydroxymethyl cellulose, hydroxypropyl methylcellulose, methylcellulose carbomer, poloxamer, polyvinyl alcohol, povidone, polyethylene oxide, carboxymethylcellulose calcium.

In one of another embodiment of the present invention, the sterile, ophthalmic pharmaceutical suspension may be aseptically sterilized using membrane filters such as PES (Polyethersulphone), PVDF (Polyvinylidene Fluoride) having pore size of about 0.45 microns to 0.22 microns to filter the suspension vehicle.

In another embodiment of the present invention, the homogenized ophthalmic suspension of brinzolamide prepared according to the process as described herein may be filled in LDPE vials of suitable capacity in volumes of 0.5 to 12 mL.

Another embodiment of the present invention consists of the active ingredient which is useful in the treatment or prevention of diseases associated with the eye(s) like elevated intraocular pressure in patients with ocular hypertension or open-angle glaucoma, ocular surface pain, uveitis, scleritis, episcleritis, keratitis, surgically-induced inflammation, endophthalmitis, iritis, atrophic macular degeneration, retinitis pigmentosa, iatrogenic retinopathy, retinal tears, retinal vein and artery occlusion, optic neuropathy, neovascular glaucoma, corneal neovascularization, cyclitis, sickle cell retinopathy, pterygium, seasonal allergic conjunctivitis, palpebral and bulbar conjunctiva, acne rosacea, superficial punctuate keratitis, herpes zoster keratitis, iritis, cyclitis, selected infective conjunctivitides, and post-operative inflammation following ocular surgery.

In another embodiment of the present invention, the active ingredient used in the sterile, ophthalmic pharmaceutical suspension, is selected from the group but are not limited to a carbonic anhydrase inhibitor (CAI), such as brinzolamide, acetazolamide, dorzolamide, methazolamide; non-steroidal anti-inflammatory drugs (NSAID), such as nepafenac, flurbiprofen, diclofenac and ketorolac tromethamine; a prostaglandin analog, such as latanoprost, travoprost, bimatoprost; a steroid, such as fluorometholone, hydrocortisone, dexamethasone, prednisolone, loteprednol, or medrysone and/or other actives used for ophthalmic formulation or a pharmaceutically acceptable salt(s), hydrate(s), solvate(s), polymorph(s), stereoisomers), ester(s), prodrug(s), complex(es) and their metabolites thereof.

In one of the preferred embodiment, the active ingredient used in the sterile, ophthalmic pharmaceutical suspension is a carbonic anhydrase inhibitor (CAI). In a preferred embodiment, the carbonic anhydrase inhibitor (CAI) is brinzolamide.

In another embodiment of the present invention, the buffers include acetates such as sodium acetate; phosphates such as sodium dihydrogenphosphate, disodium hydrogenphosphate, potassium dihydrogenphosphate and dipotassium hydrogenphosphate; s-aminocaproic acid; amino acid salts such as sodium glutamate; and boric acid and a salt thereof, wherein the buffer is generally contained in a proportion of 0.01-2.0 w/v % relative to the entire composition.

In another embodiment of the present invention, the buffer, when present, preferably has buffering capacity in the range of pH 4.5-8.5.

In another embodiment of the present invention, the pH adjusting agent preferably includes, but is not limited to, hydrochloric acid, citric acid, phosphoric acid, acetic acid, tartaric acid, sodium hydroxide, potassium hydroxide, sodium carbonate, sodium hydrogencarbonate, and combinations thereof.

In another embodiment of the present invention, the chelating agents preferably includes, but is not limited to, edetate disodium, edetate trisodium, edetate tetrasodium, diethyleneamine pentaacetate and mixtures thereof. Preferably, the chelating agent is generally present in an amount from 0.005-0.2 w/v % relative to the entire composition.

In another embodiment of the present invention, the antioxidants preferably include, but are not limited to, sodium bisulfite, potassium bisulfite, magnesium bisulfite, calcium bisulfite, sodium metabisulfite, potassium metabisulfite, calcium metabisulfite, sodium thiosulfate and sodium hydrogensulfite, ascorbic acid, sodium ascorbate, tocopherol and sulfite salts like sodium sulfite, potassium sulfite, magnesium sulfite, calcium sulfite, and combinations thereof. Preferably, the sulfite salt is generally present in an amount from 0.01-1.0% w/v relative to the entire composition.

According to one aspect of the present invention, there is provided a process for preparing sterile, ophthalmic pharmaceutical suspension, the process comprising the steps of:
a) preparing a slurry comprising brinzolamide and a surfactant;
b) adding to the brinzolamide slurry a solvent(s);
c) precipitating a portion of brinzolamide slurry as a fine particulate (preferably fine crystalline) form during cooling after autoclaving;
d) preparing sterile polymer slurry comprising polymer, tonicity agent and preservative;
e) adding said suspension vehicle of step (c) to said slurry of step (b) to obtain a suspension and making up the volume with water;
f) stirring the said suspension of step (d) while cooling.

### Examples

Following formulations are prepared to carry out DSC measurements.

Methodology:
1. Accurately weigh an appropriate quantity of sample (for brinzolamide the sample quantity taken is about 3 mg) to be examined in a sample pan.
2. The initial temperature, heating rate, and the final temperature for DSC testing of brinzolamide samples are set as:
   Initial temperature: 30° C.,
   Heating Rate: 10° C. per minute
   Final Temperature: 200° C.
3. Provide an inert environment (preferably N₂ flow) and then measure the difference in temperature between the sample and reference pan (may differ, e.g., based on the make and model of the DSC equipment).
4. The analysis is initiated and the differential thermal analysis curve is recorded.

### DSC Analysis Results—Brinzolamide Ophthalmic Suspension 1%

Sample 1 (comparative) uses crystalline brinzolamide as obtained from the manufacturer. Samples 2 and 3 are according to the present invention as shown in Table 1.

TABLE 1

| Sample | | Melting Point |
|---|---|---|
| no. | Sample details | (by DSC) |
| 1 | brinzolamide | 132.7° C. |
| 2 | brinzolamide (1%) + | 133.5° C. |
| | DMSO (0.5%) + | |
| | Polysorbate 80 (0.5%) | |
| 3 | brinzolamide (1%) + | 133.1° C. |
| | DMSO (1.0%) + | |
| | Polysorbate 80 (0.5%) | |

Regardless of formulation, there is little change in the melting point of brinzolamide, which is at about 137° C. In addition, in the present invention, the precipitating crystals have much smaller particle size.

### Powder X-Ray Diffraction (PXRD):

X'Pert PRO, X-ray diffractometer system (Pananalytical, Netherlands) was used to get powder diffraction patterns of the samples using Cu as tube anode. The diffractograms were recorded under following conditions: voltage 45 kV, 40 mA, divergence slit 10 and angular range 5 and 50° in 20. The experimental PXRD patterns were refined using X'Pert High Score software.

XRD (X-ray diffraction) uses the total X-ray scattering of a compound to determine its crystallinity and no external standard is needed. In XRD spectra every crystalline substance gives a pattern; the same substance always gives the same pattern; and in a mixture of substances each produces its pattern independently of the others. The X-ray diffraction pattern of a pure substance is, therefore, like a fingerprint of the substance. The powder diffraction method is thus ideally suited for characterization and identification of different crystalline phases. Therefore in present study we have used XRD (X-ray diffraction) as a tool to determine crystallinity of samples. It provides more accurate results and assuming that all data collection and processing variables are controlled, XRD (X-ray diffraction) provides very precise relative comparisons between samples as shown in Table 2.

TABLE 2

| | | Counts at 12.4 |
|---|---|---|
| | | (2θ) |
| S. | | value of 100% |
| No. | Experimental batches | relative intensity |
| 1 | API* | 6,000 |
| 2 | API + DMSO** (0.5%) | 30,000 |
| 3 | API + PS 80 (1%) | 30,000 |
| 4 | API + Cremophore RH 40 (0.5%) | 200,000 |
| 5 | API (1%) + DMSO (0.5%) + PS*** 80 (1%) | 74,430 |
| 6 | API (1%) + DMSO (1.5%) + PS 80 (1%) | 76,140 |
| 7 | API + Propylene Glycol (5%) | 100,000 |
| 8 | API + Polyethylene Glycol 400 (5%) | 40,000 |
| 9 | API + Malonic Acid + PS 80 | 200,000 |
| 10 | API + Ascorbic Acid (1%) | 60,000 |
| 11 | Solvent - Antisolvent Mixing | 10,000 |
| | (Acetonitrile - heptane) | |
| 12 | API + Nicotinic Acid (0.642%) | 40,000 |
| 13 | Oxalic Acid + DMSO (0.5%) | 45,000 |
| 14 | Oxalic Acid + DMSO + PS 80 (1%) | 80,000 |
| 15 | API + Malonic Acid + DMSO (0.5%) | 20,000 |
| 16 | API + Malonic Acid + DMSO + PS 80 (1%) | 200,000 |

*API is Brinzolamide.
**DMSO is dimethyl sulfoxide.
***PS 80 is Polysorbate 80 (Polyoxyethylene (20) sorbitan monooleate).

### Observation and Result:

A typical diffraction spectrum consists of a plot of reflected intensities versus the detector angle 2-THETA. IN the process PRD beam strikes at the fixed crystal plane and is diffracted. Therefore in the interpretation of XRD spectra the Position (2-THETA) value is used to identify the position of the peak with respect to the particular 20 value.

Maximum intensity (100% intensity) occurs when the beam of X-ray waves are in phase. In present study at approx. 20 value of 12.4, peak of 100% intensity is appearing in all samples. Therefore the inventors have compiled the data corresponding to this 20 angle.

Crystalline compounds will have planes going through higher electron density than the amorphous compounds therefore the corresponding intensities of former will be higher than the latter. Since a count represents the intensity and therefore the inventors of present invention conclude that the counts of crystalline compounds are higher than the amorphous compounds.

Therefore in present study the inventors have compared the counts of the different samples and determined their relative crystallinity as compared to pure API. Since the counts of these samples are higher than that of pure API the inventors of the present invention can conclude that the brinzolamide in combination with dimethyl sulfoxide (DMSO) and polysorbate 80 is more crystalline than the pure API.

It was surprisingly found by the inventors of the present invention that by the addition of certain ingredients such as dimethyl sulfoxide (DMSO) and polysorbate 80 leads to increased crystallinity of the brinzolamide. Furthermore, out of the experiments as carried by the inventors of the present invention, they surprisingly found that a formulation that comprises dimethyl sulfoxide (DMSO) and polysorbate 80 along with brinzolamide had a marked increase in the crystallinity by about 25 times to about 30 times as shown in Table 2. The crystallinity of the brinzolamide is increased from about 6000 counts to about 74,430 counts for a formulation comprising brinzolamide API (1%)+DMSO (0.5%)+PS 80 (1%) and the crystallinity of the brinzolamide is increased from about 6000 counts to about 76,140 counts for a formulation comprising brinzolamide API (1%)+DMSO (1.5%)+PS 80 (1%). The experiments further shows that the crystallinity of brinzolamide increases with the increase in the DMSO concentration.

Furthermore, the present invention also paves way to stable formulations comprising brinzolamide which may lead to an enhanced shelf life with little or no problems of resuspendability as associated with the suspension formulations due to less free energy in the system.

The inventors of present invention have furthermore found that fine particles (e.g., fine crystals) of brinzolamide were formed by using more economical methods such as by sonication technique and/or simple stirring or light homogenization, rather than ball or jet milling, wherein thus the particle size obtained in the present invention was below than about 20 microns as shown in Table 3.

During autoclaving due to high temperature and pressure, brinzolamide goes in solution. Upon completion of the autoclave cycle, during cooling, brinzolamide starts precipitating out. Brinzolamide forms needle shaped crystals of 1000 to 3000 microns. These needle shaped crystals of brinzolamide are further reduced in fine crystalline form to be used in the present invention formulation and the reduced particle size is as per table 3.

TABLE 3

| Particle size analysis | | | |
|---|---|---|---|
| Present Invention formulation | D value in micron | | |
| {API + DMSO(0.5%) + PS80(1%)} | D(0.1) | D(0.5) | D(0.9) |
| Normal cooling with stirring | 7.68 | 21.63 | 48.94 |
| Rapid cooling with stirring | 3.42 | 13.77 | 41.07 |
| Rapid cooling without stirring | 2.82 | 8.18 | 18.29 |
| with sonication 2 hr | | | |
| cooling by antisolvent mixing | 2.55 | 6.723 | 13.78 |
| with sonication 2 hr | | | |
| Normal cooling with stirring - with | 1.49 | 4.91 | 10.07 |
| 10 homogenization cycle at 1000 B | | | |
| Homogenized at 1000 bar 20 cycles | 1.11 | 4.15 | 7.78 |
| Homogenized at 1500 bar 20 cycles | 1.695 | 3.175 | 5.716 |

As shown in Table 3, dimethyl sulfoxide (DMSO) and polysorbate 80 may be added to the pure brinzolamide to reduce the particle size of the brinzolamide.

The particle size of the present formulation (API+0.5% DMSO, 1% PS 80) at various conditions were analyzed and it is observed that the D(0.9) value of present invention formulation when cooled to room temperature with stirring has reduced the particle size to 48.94. This particle size is further reduced to 41.07 through rapid cooling with stirring. This particle size is further reduced from 41.07 to 18.29 when the present formulation was rapidly cooled (with chilled water) without stirring followed by sonication for at least 2 hours. Cooling by antisolvent (solvent in which API is not soluble) addition also decreases particle size D(0.9) value to 13.78.

In order to further reduce the particle size, the inventors of the present invention further, homogenized the above formulation when normal cooled with stirring with 10 homogenization cycles at the homogenizer pressure of 1000 bar, has reduced the particle size to 10.07. It was also observed that there is a reduction of brinzolamide particle size from 10.07 to 7.78 at 1000 bar with the increase of cycles of homogenization from 10 cycles to 20 cycles.

It was further observed that as the homogenizer pressure is increased from 1000 bar to 1500 bar, the particle size decreases as depicted from the above table that the D(0.9) value dropped from 7.78 to 5.716 when the homogenizer pressure was increased from 1000 bar to 1500 bar.

The present invention is further illustrated by reference to the following examples which is for illustrative purpose only and does not limit the scope of the invention in any way.

### Formulations:

### Part One—Brinzolamide Slurry:

| | | | For 500 ml | | |
|---|---|---|---|---|---|
| Ingredients | % | mg/ml | batch (in g) | Formula 1 | Formula 2 |
| Brinzolamide | 1 | 10 | 5 | ✓ | ✓ |
| Polysorbate 80 | 0.5 | 5 | 2.5 | — | ✓ |
| Cremophor ® | 0.5 | 5 | 2.5 | ✓ | — |
| RH 40 | | | | | |
| DMSO | 2.5 | On v/v | 12.5 ml | — | ✓ |
| | | basis | | | |
| Tyloxapol | 0.025% | 0.25 | 0.125 | — | — |

### Part Two—Carbomer Slurry:

| | | | For 500 ml |
|---|---|---|---|
| Ingredients | % | mg/ml | batch (in g) |
| Carbomer 974 P | 0.45 | 4.5 | 2.25 |
| Edetate | 0.01 | 0.1 | 0.05 |
| disodium | | | |
| BKC 50% | 0.02 | 0.2 | 0.1 |
| Mannitol | 3.3 | 33 | 16.5 |
| NaCl | 0.25 | 2.5 | 1.25 |

Note: For all these formulations Carbomer slurry is prepared separately and pH is adjusted to make the same alkaline.

### Formula 1: Procedure:

Part A: Brinzolamide Slurry
1. Cremophore 40 is dissolved in hot water.
2. Brinzolamide is added in step 1 and the volume is made up to approx. 20% of batch size to form uniform slurry.
3. Autoclave the prepared slurry of step 3 for 30 min.
4. After autoclaving, cool the slurry under magnetic stirrer with moderate stirring.
5. During cooling, the slurry is homogenized to achieve uniform and required particle size.

Part B: Carbomer slurry preparation
1. In water (approx. 50% of batch size) slowly disperse the Carbomer under continuous stirring, to make solution.
2. The pH of Carbomer slurry is adjusted using 5 N NaOH solution.
   Note: During scale up this can be sterilized by in situ sterilization.

Part C: Remaining product vehicle solution
1. In water (approx. 15-20% of batch size), the sodium chloride, disodium edetate, mannitol and BKC (benzalkonium chloride) is added and dissolved.

Part D: Mixing of Part B and C
1. Filter the solution of part C (for sterilization) into part B (pre-sterilized by in situ sterilization)
2. Mix thoroughly to make the uniform solution.

Mixing of Part A & Part D
1. To the solution of part D, part A (brinzolamide slurry) is added and dissolved.
2. Mix the formulation to achieve uniform dispersion of brinzolamide slurry.
3. The pH is adjusted using NaOH solution.
4. Water is added to obtain batch volume/concentration.

### Formula 2: Procedure:

Part A: Brinzolamide Slurry
1. Polysorbate 80 is dissolved in hot water.
2. Brinzolamide is added to step 1.
3. Dimethyl sulfoxide is added to step 2 and the volume is made up to approx. 20% of batch size to form uniform slurry.
4. Autoclave the prepared slurry of step 3 for 30 min.
5. After autoclaving, cool the slurry under magnetic stirrer with moderate stirring.
6. During cooling, the slurry is homogenized to achieve uniform and required particle size.

Part B: Carbomer slurry preparation
1. In water (approx. 50% of batch size) slowly dispersed the Carbomer under continuous stirring, to make solution.
2. The pH of Carbomer slurry is adjusted using 5 N NaOH solution.
   Note: During scale up this may be sterilized by in situ sterilization.

Part C: Remaining product vehicle solution
1. In water (approx. 15-20% of batch size), the sodium chloride, disodium edetate, mannitol and BKC is added and dissolved.

Part D: Mixing of Part B and C
1. Filter the solution of part C (for sterilization) into part B (pre-sterilized by in situ sterilization)
2. Mix properly to make the uniform solution.

Mixing of Part A & Part D
1. To part D, part A (brinzolamide slurry) is added and dissolved.
2. Mix the formulation to achieve uniform dispersion of brinzolamide slurry.
3. The pH is adjusted using NaOH solution.
4. Batch volume was made up with water.

1-13. (canceled)

14. An aqueous composition comprising water, particles of brinzolamide, a polar organic solvent, and a non-ionized non-aromatic ethoxylated surfactant.

15. The composition of claim 14 wherein the particles of brinzolamide have an average size less than 50 μm after crystallization and before optional particle size reduction.

16. The composition of claim 14 wherein the polar organic solvent comprises DMSO.

17. The composition of claim 14 wherein the surfactant comprises at least one of polysorbate 80 or polyethoxylated castor oil.

18. The composition of claim 14 which is a sterile suspension suitable for ophthalmic use.

19. The composition of claim 14 which further comprises one or more of a polymer, an isotonicity agent, a viscosity enhancing agent, a buffer, a pH adjusting agent, an antioxidant, a chelating agent, and a preservative.

20. The composition of claim 14, wherein the composition does not comprise tyloxapol or Triton X.

21-29. (canceled)

30. A method of treating an ocular condition treatable with brinzolamide, in a person in need thereof, comprising administering to the person an ophthalmic composition comprising water, an effective amount of fine particles of brinzolamide, a polar organic solvent, and a non-ionized non-aromatic ethoxylated surfactant.

31. The method of claim 30 wherein the condition treatable with brinzolamide includes elevated intraocular pressure.

32. The method of claim 30 wherein the brinzolamide is administered one drop per eye, 1-3 times per day, at a concentration of 1-15 mg brinzolamide per ml of the ophthalmic composition.

33. The method of claim 30 wherein the particles of brinzolamide have an average size less than 20 μm.

34. The method of claim 30 wherein the polar organic solvent comprises DMSO.

35. The method of claim 30 wherein the surfactant comprises at least one of polysorbate 80 or polyethoxylated castor oil.

36. The method of claim 30 wherein the ophthalmic composition is a sterile suspension suitable for ophthalmic use.

37. The method of claim 30 wherein the ophthalmic composition further comprises one or more of a polymer, an isotonicity agent, a viscosity enhancing agent, a buffer, a pH adjusting agent, an antioxidant, a chelating agent, and a preservative.

38. The method of claim 30, wherein the ophthalmic composition does not comprise tyloxapol or Triton X.

39-45. (canceled)

46. The method of claim 30 wherein D(0.9) of the fine particles is less than 15 μm when measured by a Coulter counter.

47. The composition of claim 15 wherein the surfactant comprises at least one of polysorbate 80 or polyethoxylated castor oil.

48. The composition of claim 47 which is a sterile suspension suitable for ophthalmic use.

49. The composition of claim 48 wherein the polar organic solvent comprises DMSO.
